(19) United States
(12) Patent Application Publication
(10) Pub. No.: US 2011/0034371 A1
(43) Pub. Date: Feb. 10, 2011

(54) L. CASEI RHAMNOSUS SECRETED FACTORS AND USE THEREOF

(75) Inventors: Kou-Cheng Peng, Taoyuan (TW); Yi-Han Chiu, Hualien Country (TW)

Correspondence Address: WPAT, PC;INTELLECTUAL PROPERTY ATTORNEYS, 2030 MAIN STREET, SUITE 1300, IRVINE, CA, 92614, US

(73) Assignees: Kou-Cheng Peng, Taoyuan (TW); Yi-Han Chiu, Hualien Country 973 (TW); Jun-Song Lin, Hualien County (TW)

(21) Appl. No.: 12/536,806

(22) Filed: Aug. 6, 2009

(51) Int. Cl. A61K 38/02; C07K 2/00; A61P 1/00

(52) U.S. Cl. 514/2.4; 530/300; 514/18.9

(57) ABSTRACT

The present invention relates to a composition for inhibiting gastrointestinal inflammation comprising an effective amount of Lactoacillus casei rhamnosus secreted factors 5 to 30K fraction.

### FIELD OF THE INVENTION

The present invention relates to a composition for inhibiting gastrointestinal inflammation comprising an effective amount of Lactoacillus casei rhamnosus secreted factors 5 to 30K fraction.

### BACKGROUND OF THE INVENTION

Probiotics have been evaluated as an alternative and safe therapeutic approach for gastrointestinal inflammation (Kruis et al., 2004; Bibiloni et al., 2005).

Several clinical trials have suggested that treatment with a multispecies probiotic, VSL#3, including three Bifidobacterium species, four Lactobacillus species and S. salivarius ssp. thermophilus, was considered effective in maintaining remission and decreasing relapse rate in gastrointestinal inflammation patients (Miele et al., 2009). In two studies the treatment with Escherichia coli Nassle 1917 had been shown remission maintenance of ulcerative colitis (UC) (Kruis et al., 2004). However, conflicting results have been observed for various probiotic strains in clinical uses. Treatment with either L. rhamnosus GG (LGG) or L. johnsonii (LA1) was incapable of improving the clinical conditions of Crohn's disease (CD) or preventing endoscopic recurrence of CD, respectively (Kuisma et al., 2003; Bousvaros et al., 2005; Marteau et al., 2006; Van Gossum et al., 2007).

Previous investigation had demonstrated that the Lcr can control bacterial and gastrointestinal disease (Lin et al., 2009) so as to initiate this investigation of effectiveness of the Lcr in host immune mediation.

### SUMMARY OF THE INVENTION

The present invention relates to a composition for inhibiting gastrointestinal inflammation comprising an effective amount of Lactoacillus casei rhamnosus secreted factors 5 to 30K fraction.

The present invention further relates to wherein the secreted factors of L. casei rhamnosus alters pro-inflammatory or anti-inflammatory cytokines balance to treat gastrointestinal inflammation.

The present invention also relates to a method of suppressing pro-inflammatory cytokine production comprising administrating a subject an effectively amount of the extract of Lactoacillus casei rhamnosus secreted factors 5 to 30K fraction.

The present invention further relates a method of suppressing activation and proliferation of inflammatory cells comprising administrating a subject an effectively amount of the extract of Lactoacillus casei rhamnosus secreted factors 5 to 30K fraction.

### BRIEF DESCRIPTION OF THE DRAWINGS

FIG. 1 shows strain-dependent promotion of apoptosis in THP-1 cells in the absence or presence of probiotic-derived secreted factors. The results represent typical donors (A) and mean±standard error of the mean (B) of three different experiments.

FIG. 2 shows partial characterization of factors promoting THP-1 apoptosis. (A) THP-1 cells were stimulated with 25 μg mL⁻¹ four different fractions. (B) THP-1 cells were stimulated with 30 mins boiled, 1 mg mL⁻¹ trypsin or proteinase K treatment of the LcrS₅₋₃₀.

FIG. 3 shows THP-1 cells (A-B) and HT-29 cells (C) were treated with various concentrations of the LcrS₅₋₃₀ or 10 μM Taxol as positive control for 24 h. (A) The TUNEL staining was observed by light microscope. Arrows indicated representative apoptotic cells. (B) The percentage of cells undergoing apoptosis from a representative experiment. (C) The apoptotic HT-29 cells exposure to the LcrS₅₋₃₀ was determined by Annexin V-FITC and PI double staining followed by flow cytometric analysis.

FIG. 4 shows incubation with the LcrS₅₋₃₀ increases the numbers of Annexin V positive cells in a time- and dose-dependent manner. 1×10⁶ lymphocytes, monocytes or THP-1 cells per test were collected after incubation with the LcrS₅₋₃₀ for various time intervals (A) and in dose-ranging experiments with 3.125-25 μg mL⁻¹ (B). Cell apoptosis was determined by double staining with Annexin V-FITC and PI.

FIG. 5 shows cytofluorometric analysis of mitochondrial membrane potential (ΔΨm). (A) One representative analysis of ΔΨm in monocytes stimulated by various concentrations of the LcrS₅₋₃₀. (B), JC-1 red fluorescence/JC-1 green fluorescence ratio of lymphocytes, monocytes and THP-1 with depolarized ΔΨm with or without the LcrS₅₋₃₀ for various time intervals.

FIG. 6 shows the LcrS₅₋₃₀ cause decrease in Bcl-2 expression, increase in Bax and caspase 9 expressions, cytochrome c (Cyt c) release and caspase 3 activation. The mRNA was detected by RT-PCR (A), and the protein levels were determined by western blot (B). Both the mRNA and protein expression of actin were internal controls. (C), Cells were incubated with FITC-conjugated anti-active caspase-9 antibody and analysis by flow cytometry.

FIG. 7 shows inhibition of cytokine production by the LcrS₅₋₃₀ in lymphocytes, monocytes and THP-1 cells. Cell culture bioassay were performed by co-incubating lymphocytes, monocytes or THP-1 cells with E. coli-derived LPS and the 25 μg mL⁻¹ LcrS₅₋₃₀. Human IL-1beta (A), IL-6 (B), IL-8 (C) and TNF-alpha (D) cytokine were determined by sandwich ELISA in culture supernatants following cell culture bioassay.

FIG. 8 shows The LcrS₅₋₃₀ induced TGF-β1 production and promoted TGF-β independent apoptosis. (A), Human TGF-β1 quantities were determined by TGF-01 specific ELISA in culture supernatants following lymphocytes, monocytes or THP-1 cells of cells culture bioassay. (B), THP-1 cells were treated with TGF-β1 or the LcrS₅₋₃₀ in the absence or in the presence of 1 μg mL⁻¹ TGF-β inhibitor, SB431542, for 24 h.

### DETAILED DESCRIPTION OF THE INVENTION

In this invention, several Lactobacillus strains were investigated for the differential abilities in promoting apoptosis on THP-1 cells. The Lcr exhibited more potent apoptosis inducing ability than the L. casei and the L. rhamnosus strains. Probiotics, as preventive or therapeutic agents against gastrointestinal inflammation, are attractive and alternative approach for the attenuation of mucaosal inflammation. Many clinical studies over the last decades demonstrated that protiotic species possessed beneficial effects to the gastrointestinal inflammation. However, inconsistency existed might result from differences in probiotic species/strains. These inconsistencies drew the attention to understand the mechanisms of probiosis by specific strains for possible clinical applications.

Initial characterization of the apoptosis inducing factor(s) in the LcrS employed the MWCO ultrafiltration. The apoptosis inducing factor(s) were presented in the 5-30 kDa fraction. Several studies have described apoptosis promotion via factor(s) produced by probiotic such as human breast cancer cells with fermented soy milk; human γδ T cell with E. coli Nissle 1917 supernatant; and in human myeloid leukemia-derived cells with L. reuteri. The identities of these factor(s) have not been deciphered up till now that they are the Lcr derived small factor(s). This apoptosis inducing factor(s) are heat stable proteins or peptides components as results of the heat inactivation and the protease treatments experiments.

The pathological feature of gastrointestinal inflammation is characterized by a high density of mucosal cells within the inflamed tissues that mainly consist of activated T cells, peripheral blood neutrophils and monocytes/macrophages. Extensive studies in recent years have shown activation and increased survival time on leucocytes might contribute to severity of intestinal inflammation and clinical relapse in both CD and UC. Activation-induced cell death (AICD) is an important mechanism to limit the number of active monocyte/lymphocyte and to terminate the immune response. The LcrS₅₋₃₀ was effective in promoting lymphocyte/monocyte/THP-1 cell apoptosis in a dose- and time-dependent manners. Despite the paucity of data regarding probiotics in promoting apoptosis of immune effector cells as an alternative gastrointestinal inflammation therapy, the experiments of the γδ T cell going through programmed cell death by E. coli Nissle and human myeloid leukemia-derived cells proceeding TNF-induced apoptosis by L. reuteri provided a rational basis for apoptotic strategy in gastrointestinal inflammation therapy. Our study in monocytes showed that 12.5 μg mL⁻¹ and 25 μg mL⁻¹ of the LcrS₅₋₃₀ could disrupt the mitochondrial membrane potential and proceeded apoptotic cell death within 3 hrs following exposure to 25 μg mL⁻¹ of the LcrS₅₋₃₀. Moreover, the increase of Bax/Bcl-2 ratio, release of cytochrome c, and activation of caspases 9 and caspase 3, showed the LcrS₅₋₃₀ is capable of inducing the monocytes apoptosis via mitochondria pathway.

The anti-TNF antibody (infliximab) is the FDA approved treatment for CD and UC. Several studies demonstrated that the infliximab induced monocytes apoptosis and lost of CD68+ monocytes as well as CD4+ and CD8+ T lymphocytes in the lamina propria. Also, the infliximab exerted its killing activity on human peripheral blood T cells by as much as 50.6% after 18 h cultured with the 5 μg mL⁻¹ infliximab. In our study, the LcrS₅₋₃₀ promoted apoptosis of lymphocytes, monocytes and THP-1 cells by as much as 93%, 97% and 74%, respectively. The induction of apoptosis did not require the Fas/FasL signal transduction pathway, but rather involved upregulation of the Bax/Bak followed by mitochondrial release of cytochrome c. Interestingly, the extent of immune effector cells apoptosis inducing mechanism by the infliximab and probiotic Lcr were similar. Plus the safety history of the probiotics, the Lcr could be a useful adjunctive treatment for gastrointestinal inflammation patients.

The loss of intestinal epithelial cell (IEC) function and subsequent changes in epithelial tight junction protein expression and IEC apoptosis was also a critical component in the initiation and perpetuation of the gastrointestinal inflammation. Probiotics have been used to investigate protective effect by regulate IEC survival for treating and preventing intestinal inflammation. In vitro study indicated that LGG reduced intestinal epithelial apoptosis through upregulating expression of anti-apoptotic and cytoprotective genes. Also, soluble factors from LGG stimulating anti-apoptotic Akt activation and preventing cytokine-mediated apoptosis. In this research the LcrS₅₋₃₀ was quite unique promoted apoptosis of monocyte-like cells but not IEC. The result was consistent with prior studies that probiotics can protect intestinal integrity from promoting survival of IEC.

The LcrS₅₋₃₀ possessed potent inhibitor(s) for LPS induced pro-inflammatory cytokines, such as IL-1β, IL-6, TNF-α, and chemokine, IL-8, but not in IFN-γ and IL-12 (Data not shown). The suppression of IL-1β, IL-6, TNF-α, and IL-8, suggested that the LcrS₅₋₃₀ concomitantly suppress lymphocyte/monocyte/THP-1 chemotaxis and cellular activation. TNF-α is a crucial proinflammatory cytokine in various inflammatory disorder. Blocking of TNF-α is efficient in the treatment of patients with CD and UC. Interestingly, plenty studies indicated that apoptosis was indeed lined to clinical efficacy to various anti-TNF agent. TNF-α inhibitors, such as the infliximab and the adalimumab, have shown clinical efficacy in gastrointestinal inflammation and exerted strong apoptotic effects. However, the other anti-TNF agent, etancercept, was not able to establish a positive influence on the course of CD due to failed apoptosis inducing ability in monocytes and lymphocytes. Our data indicated that the LcrS₅₋₃₀ possessed both TNF-α suppression and apoptosis promotion abilities as the infliximab.

TGF-β is the potent anti-inflammatory cytokine and has a vital role in suppressing the activation and proliferation of inflammatory cells. Also, TGF-β has been implicated an essential role in disease remission by promoting maturation of intestinal epithelial cells and healing in wounds and ulcers. The L. paracasei has been reported to induce populations of regulatory CD4+ T cells which produce high levels of the modulatory cytokines, IL-10 and TGF-β. The Lactobacilli modulate cytokine production in bone-marrow-derived dendritic cells with a net effect of altering overall cytokine profiles in a specifies-dependent manner. A study also showed that pediatric gastrointestinal inflammation patients in remission compared to those with active disease had higher levels of TGF-β1. In this study, that the LcrS₅₋₃₀ can induce high TGF-01 production, but not IL-10, in lymphocytes, monocytes, and THP-1 cells implied the LcrS₅₋₃₀ altered the balance between pro-inflammatory and anti-inflammatory cytokines and highlight their important immunomodulatory roles in inflammatory diseases.

TGF-β could trigger apoptosis in myeloid leukemia cells. The SB-431542, a specific inhibitor of TβRI, inhibits the TGF-β-induced apoptosis in several cell types by blocking TGF-β signaling. Upregulated TGF-β production could promote monocytes apoptosis and contributed to the prevention of tissue injury. It was important to confirm the consequence of TGF-β production in regulating the apoptosis of monocytes following the LcrS₅₋₃₀ treatment. The SB-431542 significantly blocked TGF-β1- and 12.51 g mL⁻¹-induced apoptosis. The higher dose of LcrS₅₋₃₀ induced a significant amount of apoptosis in THP-1 cells, but this was complete inefficiently abrogated by the SB 431542. So, the LcrS₅₋₃₀ could trigger TGF-β independent apoptosis.

In conclusion, the probiotic L. casei rhamnosus produces heat-stable molecules with MW range between 5-30 kDa, mainly proteins that promoted lymphocyte, monocyte and THP-1 cell apoptosis via mitochondrial pathway without affecting intestinal epithelial cells. The LcrS₅₋₃₀ also inhibited LPS-induced inflammatory cytokines in activated immune cells. These findings suggested that the LcrS₅₋₃₀ could be a pharmacologic agent in gastrointestinal inflammation therapy.

### EXAMPLES

### Exp. 1

### The Method of Preparing LcrS₅₋₃₀

The Lcr were grown at 37° C. in MRS broth (Difco, Sparks, Md., USA) for 48 h until stationary phase. The Lcr cell-free supernatant was collected by centrifugation at 1,000×g for 10 min at 4° C. Fresh MRS broth used as the control in apoptosis and the cytokines assay was subjected to similar treatment.

The cell-free supernatant was collected and centrifuged in filter device (Amicon Ultra-15; Millipore, Bedford, Mass., USA) with MWCO 50 kDa, 30 kDa and 5 kDa ultrafiltration membranes. The supernatant loaded into 50 kDa ultrafiltration device, 3300×g for 30 minutes. The concentrate was the >50 kDa fraction. Subsequently, the <50 kDa fraction was centrifuged at 3300×g for 30 min in 30 kDa ultrafiltration membrane. The upper layer is the 30-50 kDa fraction. The 5-30 kDa fraction was obtained by applying the <30 kDa fraction to 5 kDa ultrafiltration device. The filtrate was the <5 kDa fraction. Each fraction was filtrated through 0.22 μm membrane (Millipore) and kept at 4° C. before used. The protein concentration was determined by the BCA protein assay kit (Pierce, Rockford, Ill., USA) following the manufacture's instruction.

The supernatant was mixed with 1 mg mL⁻¹ trypsin or proteinase K (Sigma-Aldrich Co. Ltd. Poole, UK) at room temperature overnight. The supernatant was treated with heat by incubating supernatants at 100° C. for 30 minutes prior to intended assay.

THP-1 cells and HT-29 cells were obtained from the Bioresource Collection and Research Center (Hsinchu, Taiwan). The THP-1 cells were maintained in Falcon 100×20 mm plastic culture flasks (Becton Dickinson Labware, Oxnard, Calif., USA) in RPMI-1640 medium (Gibco, Invitrogen, Paisley, UK), supplemented with 10% fetal bovine serum (Gibco, Invitrogen), L-glutamine (200 mmol L⁻¹), 2-mercapto-ethanol (5×10⁻⁵ mol L⁻¹) (Sigma-Aldrich), 0.1 mg of streptomycin sulfate (Sigma-Aldrich) per mL, 0.1 mg of penicillin (Brittania Pharmaceuticals Ltd., Poole, UK) per mL. The HT-29 cells was grown in Dulbecco's modified Eagle's medium (Gibco, Invitrogen) supplemented with 10% fetal bovine serum. All cells were incubated at 37° C. in a humidified atmosphere containing 5% CO₂ for various times.

Peripheral blood mononuclear cells (PBMCs) were prepared by density gradient centrifugation of blood over Histopaque-1077 (Sigma-Aldrich) and washed with phosphate buffered saline (PBS) (Gibco, Invitrogen) twice prior to use. For studies requiring purified monocytes, PBMCs were depleted of T cells, B cells, NK cells and dendritic cells by labeling with specific biotin-conjugated antibodies cocktails and anti-biotin MicroBeads in a monocyte isolation kit (Miltenyi Biotec GmBH, Bergisch Gladbach, Germany) used according to the manufacturer's instructions. The magnetically labeled nonmonocytic cells were depleted by retention on an LS MACS column in a magnetic field generated by a MACS separator (Miltenyi Biotech GmBH), which allowed the unlabeled monocytes to be collected. The purity of monocyte preparation was >95%. Each cell preparation was aliquoted to contain 1×10⁶ cells mL⁻¹ in RPMI 1640 medium (Gibco, Invitrogen) supplemented with 10% fetal calf serum (Gibco Invitrogen), 0.1 mg of streptomycin sulfate (Sigma-Aldrich) per mL, 0.1 mg of penicillin (Brittania Pharmaceuticals Ltd) per mL, and 100 mM L-glutamine (Sigma-Aldrich). Cell cultivation was 37° C. in a humidified atmosphere containing 5% CO₂ for various times.

### Exp. 2

### Probiotic Lcr Preferentially Induced Apoptosis in Human Monocytic Leukemia-Derived Cell-Lines

Lymphocytes, monocytes and THP-1 cells (10⁶ per test) were collected after incubation with LcrS, 3.125-25 μg mL⁻¹, for various times. After washing twice with PBS, the cells were resuspended in Annexin V binding buffer (10 mM HEPES-NaOH (pH 7.4), 140 mM NaCl, and 2.5 mM CaCl₂). After centrifugation, the cells were incubated in 500 μl of the same buffer containing 1.25 it FITC-conjugated Annexin V (BD PharMingen, San Diego, Calif., USA) at room temperature for 15 min in the dark. Data acquisition and analysis were performed by the Becton-Dickinson FACSan flow cytometer (Franklin Lakes, N.J., USA) using CellQuest software (Becton-Dickinson, Oxford, UK).

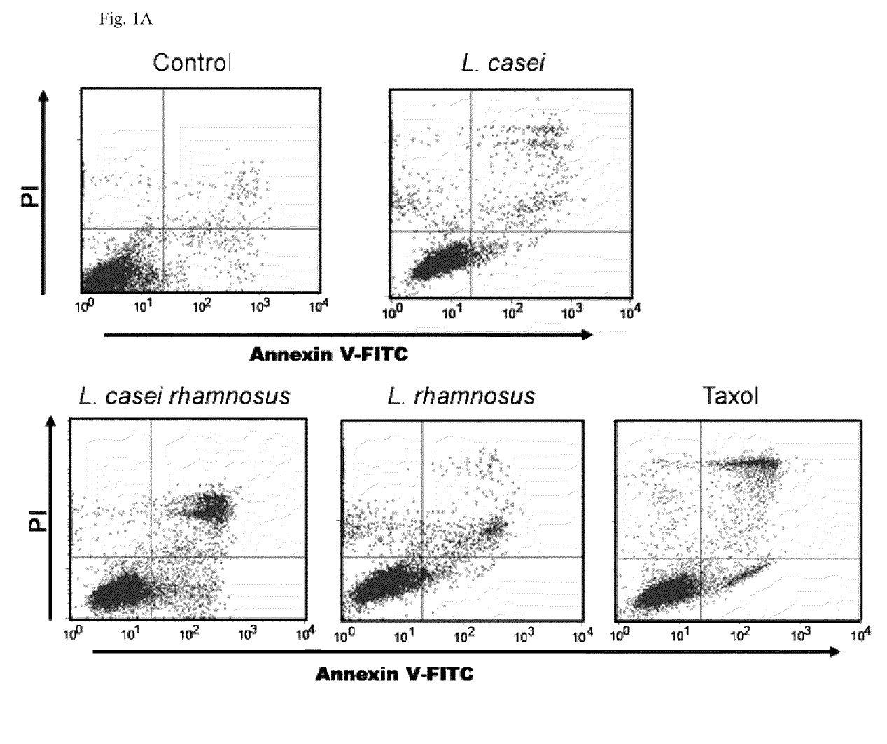

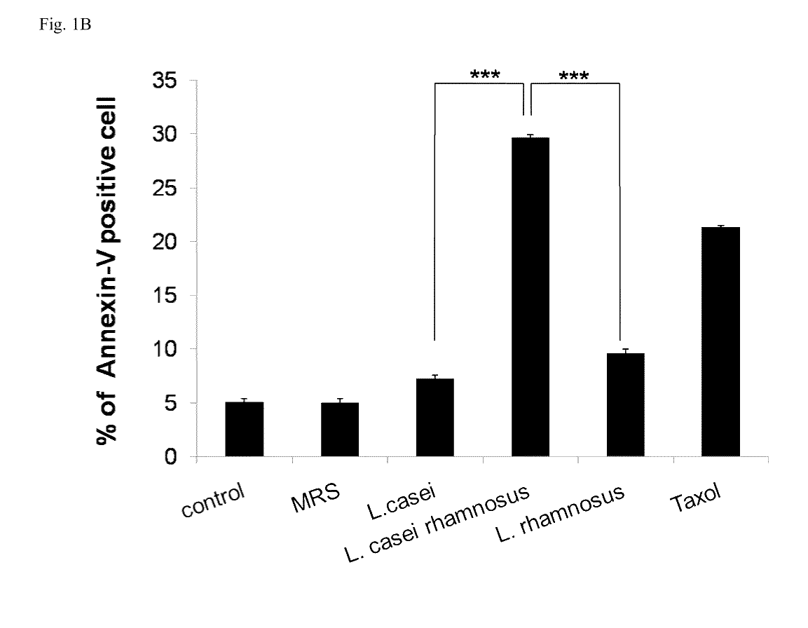

Following coincubating THP-1 cells with Lactobacillus conditioned media, the relative apoptosis promoting activities of probiotics were evaluated by Annexin-V FITC and PI staining followed by the flow cytometry in cell-free culture supernatants. The ability and specificity of Lactobacillus strains' supernatants induced the THP-1 cell apoptosis were determining by culturing the cells with each 25 μg mL¹ supernatant for 24 h. The LcrS increased 24.58% Annexin-V positive cells (P<0.001), whereas the L. casei and L. rhamnosus strains performed a limited Annexin-V positive inducing ability (2.17% and 4.53%, respectively) (FIG. 1).

### Exp 3

### Partial Characterization of the Lcr Apoptosis Inducing Factor(s)

Lymphocytes, monocytes and THP-1 cells (10⁶ per test) were collected after incubation with LcrS, 3.125-25 μg mL⁻¹, for various times. After washing twice with PBS, the cells were resuspended in Annexin V binding buffer (10 mM HEPES-NaOH (pH 7.4), 140 mM NaCl, and 2.5 mM CaCl₂). After centrifugation, the cells were incubated in 500 W of the same buffer containing 1.25 μl FITC-conjugated Annexin V (BD PharMingen, San Diego, Calif., USA) at room temperature for 15 min in the dark. Data acquisition and analysis were performed by the Becton-Dickinson FACSan flow cytometer (Franklin Lakes, N.J., USA) using CellQuest software (Becton-Dickinson, Oxford, UK).

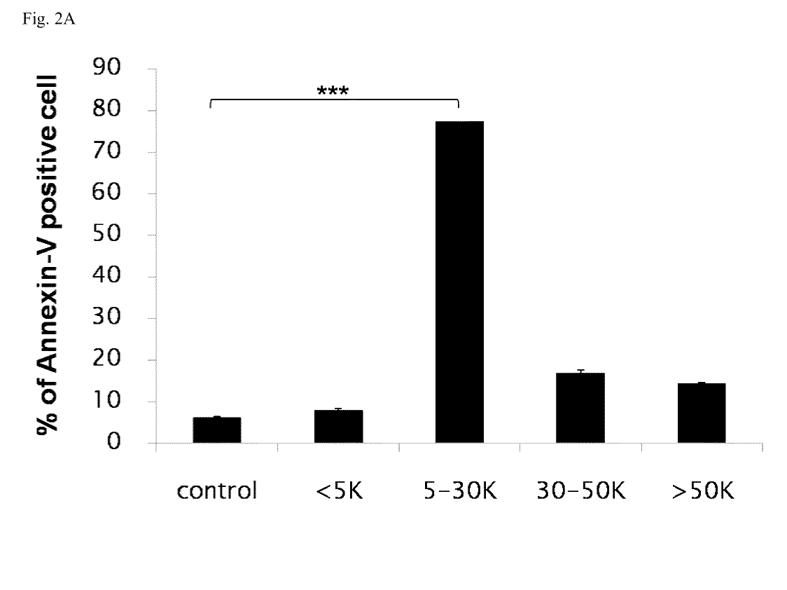

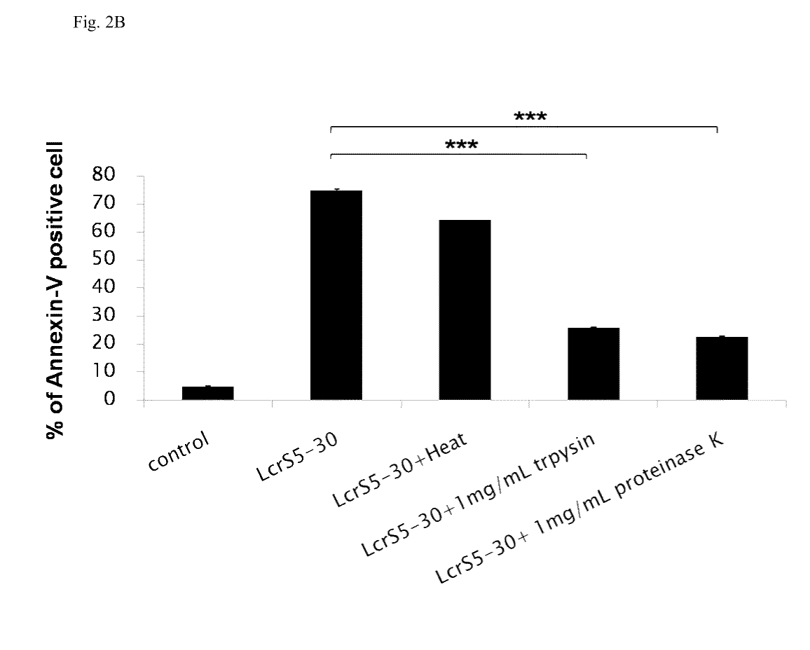

The LcrS was divided into <5 kDa, 5-30 kDa, 30-50 kDa and >50 kDa four fractions with MWCO 50 kDa, 30 kDa and 5 kDa ultrafiltration membranes and approximately 8.03%, 77.36%, 16.91% and 14.43%, respectively, of Annexin-V positive cells were induced. The LcrS₅₋₃₀ promoted apoptosis more efficient than others (P<0.001) (FIG. 2A). Then, the LcrS₅₋₃₀ was subjected to various treatments in order to identify the nature of the compounds responsible for the promotion of apoptosis of THP-1 cells. Heating did not decrease the effect of apoptosis promotion, suggesting that apoptotic promoting compounds were heat-stable. Both proteinase K and typsin treatments reduced 69.8% and 65.53%, respectively, of Annexin-V positive cells comparing to untreated fraction (P<0.001). These results suggested that proteinaceous compounds or domain(s) may involve in the apoptotic promotion process (FIG. 2B).

### Exp. 4

### Selective Apoptosis Promotion on Monocytes but not Intestinal Epithelial Cells by the LcrS₅₋₃₀

Lymphocytes, monocytes and THP-1 cells (10⁶ per test) were collected after incubation with LcrS, 3.125-25 μg mL⁻¹, for various times. After washing twice with PBS, the cells were resuspended in Annexin V binding buffer (10 mM HEPES-NaOH (pH 7.4), 140 mM NaCl, and 2.5 mM CaCl₂). After centrifugation, the cells were incubated in 5001 of the same buffer containing 1.25 μl FITC-conjugated Annexin V (BD PharMingen, San Diego, Calif., USA) at room temperature for 15 min in the dark. Data acquisition and analysis were performed by the Becton-Dickinson FACSan flow cytometer (Franklin Lakes, N.J., USA) using CellQuest software (Becton-Dickinson, Oxford, UK).

THP-1 cells were cultured in plates and treated with 10 μM taxol or 3.125-25 μg mL⁻¹ of the LcrS₅₋₃₀ for 24 hrs. After being washed with PBS, 1×10⁶ cells were fixed with 4% formaldehyde in PBS for 10 min at room temperature and permeabilized with 80% ethanol at −20° C. DNA fragments of apoptotic cells were labeled by 57 μL biotin and 3 μL catalyzed by terminal deoxynucleotidyl transferase (TdT) per sample for 2 h. Biotinylated nucleotides were detected using 100 μL streptavidin-horseradish peroxidase (HRP) conjugate. Diaminobenzidine reacted with the labeled sample to generate an insoluble colored substrate at the site of DNA fragmentation. Slides were counterstained with methyl green to assist in the morphological evaluation and characterization of normal and apoptotic cells and the cells were observed by light microscope. A total of 200 cells per sample were analyzed, and the numbers of apoptotic cells were expressed as percentages of the total cells.

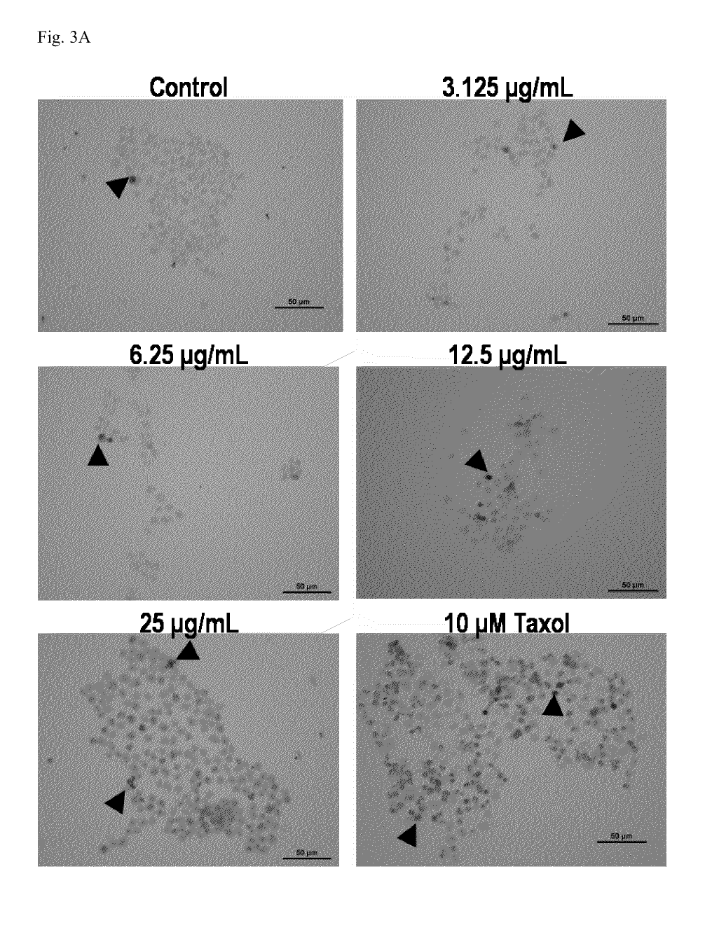

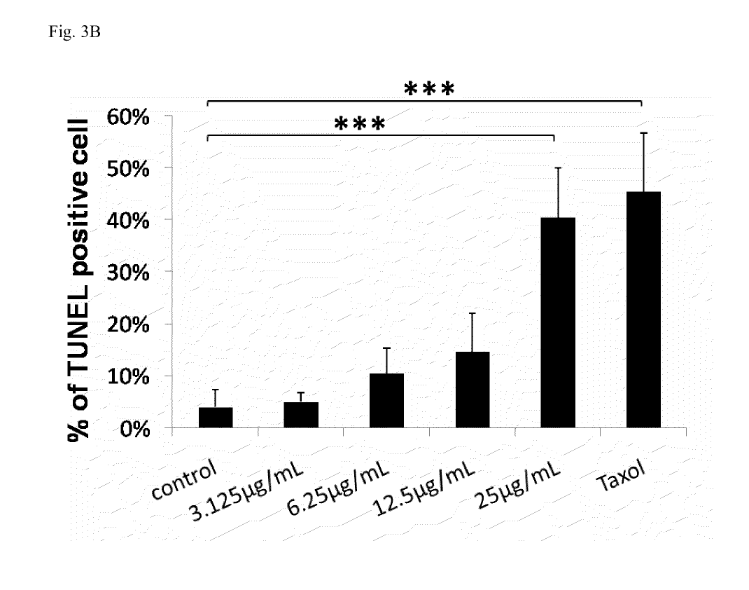

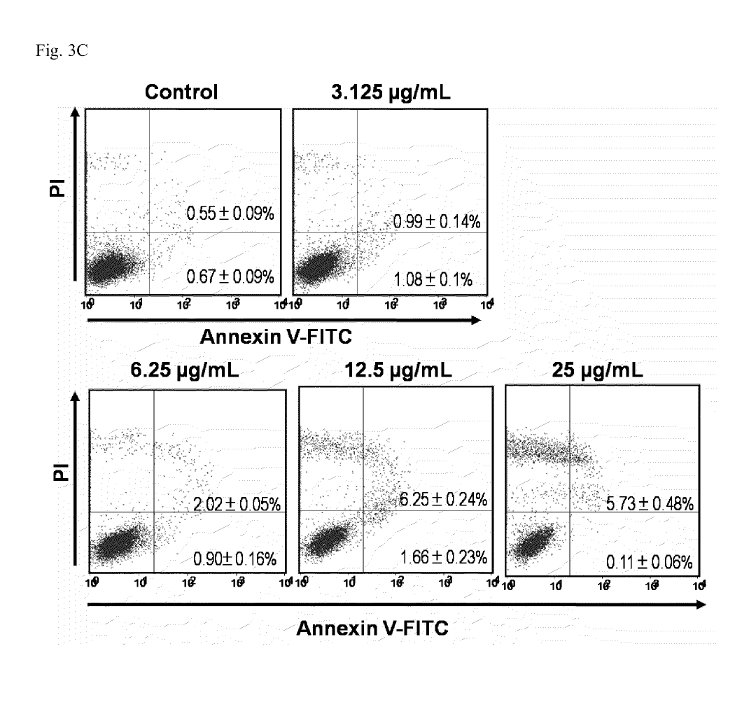

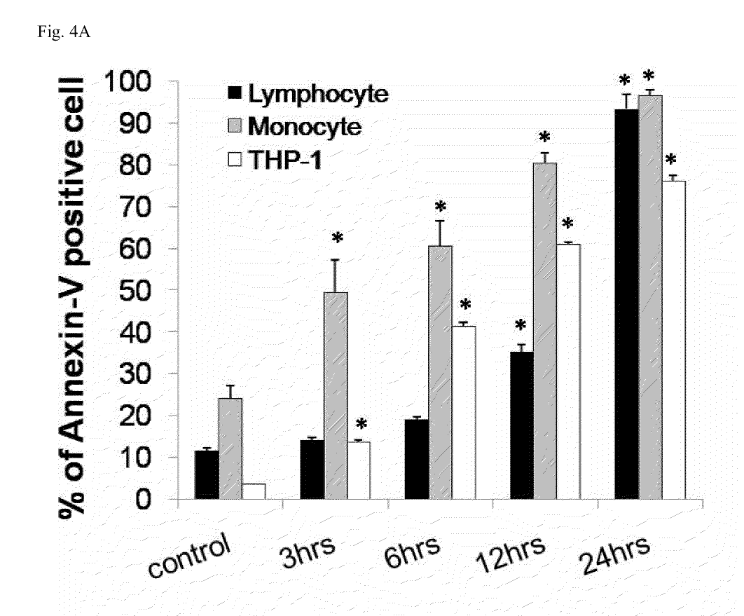

THP-1 cells were analyzed by the TUNEL assay, while human colonic epithelial carcinoma cell line (HT-29) cells were analyzed by Annexin V-FITC and PI double staining by the flow cytometry. Exposure of THP-1 cells to various concentrations of the LcrS₅₋₃₀ (3.125 to 25 μg mL⁻¹) led to apoptosis in a dose-dependency (FIGS. 3A & 3B). By contrast, the majority of HT-29 cells did not undergo apoptosis as exposure to the same concentration of the LcrS₅₋₃₀ (FIG. 3C).

### Exp. 5

### Dose and Time Dependency of the LcrS₅₋₃₀-induced Cytotoxicity

Lymphocytes, monocytes and THP-1 cells (10⁶ per test) were collected after incubation with LcrS, 3.125-25 μg mL⁻¹, for various times. After washing twice with PBS, the cells were resuspended in Annexin V binding buffer (10 mM HEPES-NaOH (pH 7.4), 140 mM NaCl, and 2.5 mM CaCl₂). After centrifugation, the cells were incubated in 500 μl of the same buffer containing 1.25 μl FITC-conjugated Annexin V (BD PharMingen, San Diego, Calif., USA) at room temperature for 15 min in the dark. Data acquisition and analysis were performed by the Becton-Dickinson FACSan flow cytometer (Franklin Lakes, N.J., USA) using CellQuest software (Becton-Dickinson, Oxford, UK).

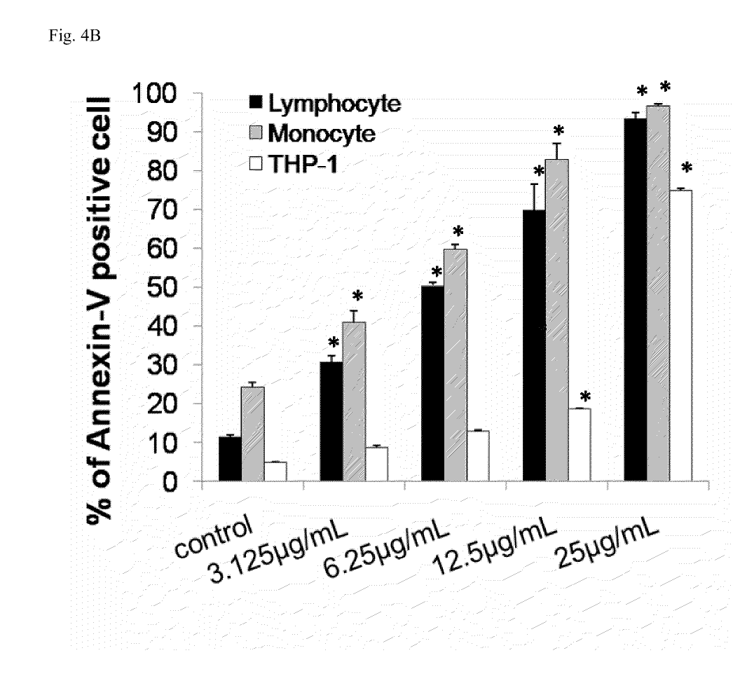

In order to quantify apoptosis induction on immune cells by the LcrS₅₋₃₀, both monocytes and lymphocytes were purified from PBMC sample and subjected to similar treatment describe in experimental procedure. Double staining of Annexin-V FITC and PI confirmed the time and dose dependent apoptosis promotion on lymphocytes/monocytes/THP-1 cells by the LcrS₅₋₃₀. To determine the timing of apoptosis inducing by the LcrS₅₋₃₀, cultured cells were treated 25 μg mL⁻¹ LcrS₅₋₃₀ for various time intervals. The flow cytometry analyses showed that the LcrS₅₋₃₀ induced monocytes and THP-1 apoptosis in time-dependent manner. The LcrS₅₋₃₀ also promoted apoptosis of lymphocytes at 12 to 24 h after 25 kg mL⁻¹ treatment (FIG. 4A). To determine the dose of LcrS₅₋₃₀ required to induce apoptosis, cells were treated with different concentrations of the LcrS₅₋₃₀ for 24 h. The results showed that 3.125 μg mL⁻¹ of the LcrS₅₋₃₀ induced apoptosis on monocytes and lymphocytes effectively. Whereas, the 25 μg mL⁻¹ LcrS₅₋₃₀ promoted apoptosis of THP-1 cells significantly (FIG. 4B).

### Exp. 6

### Apoptosis of Monocytes by the LcrS₅₋₃₀ Via Mitochondrial Pathway

ΔΨm was measured by using the lipophilic cation JC-1, which potential-dependent accumulation in mitochondria, indicated by a fluorescence emission shift from green (JC-1 at monomeric form, 527 nm) to red (JC-1 at aggregative form, 590 nm). Consequently, mitochondrial depolarization is indicated by a decrease in the red/green fluorescence intensity ratio. Purified lymphocytes, monocytes and THP-1 cells were divided into aliquots containing 5×10⁵ cells mL⁻¹ in supplemented RPMI medium and incubated with or without LcrS₅₋₃₀ for various time intervals and in dose-ranging experiments from 3.125-25 μg mL⁻¹ at 37° C. in the presence of 5% CO₂. Cells were then washed twice with PBS prior to stain with 1 μM JC-1 in dimethyl sulfoxide (DMSO) (Gibco Invitrogen) for 30 min in the dark at 37° C., and immediately analyzed on the Becton-Dickinson FACSan flow cytometer with 488 nm excitation.

Lymphocytes, monocytes and THP-1 cells (5×10⁵ cells n-L⁻¹) were collected after incubation with LcrS₅₋₃₀ for various time intervals and in dose-ranging experiments from 3.125-25 μg mL⁻¹ at 37° C. in the presence of 5% CO₂. After incubation the cells were fixed with 4% formaldehyde in PBS for 5 min at room temperature, and this was followed by permeabilization with the saponin buffer (0.04% saponin, 50 mM glucose, 0.1% sodium azide) in PBS. Intracellular active caspase-9 subunits were detected by incubation with FITC-conjugated anti-active human caspase-9 antibody (BD Pharmingen) in the presence of rabbit serum (Sigma-Aldrich) to block nonspecific binding. Substrate cleavage to release free FITC fluorescence intensities were recorded in the Becton-Dickinson FACS-Calibur flow cytometer with excitation wavelength set at 488 nm and emission wavelength at 520 nm.

Monocytes and THP-1 cells were harvested from cultures after treatment with the LcrS₅₋₃₀ for various time intervals, 3-24 h. Cells were lysed with a buffer containing 1% Triton X-100, 50 mM Tris (pH 7.5), 10 mM EDTA, 0.02% NaN₃, and a protease inhibitor mixture (Sigma-Aldrich). After being freeze-thawed once, cell lysates were centrifuged at 14,000×g for 30 min at 4° C. The supernatants were collected and boiled in the sample buffer for 5 min. After SDS-PAGE, proteins were transferred to polyvinylidene difluoride membrane (Millipore), blocked overnight at 4° C. in PBS-T (PBS plus 0.5% Tween 20) containing 10% skim milk, and probed with Abs against cytochrome c, pro-caspase 3, active caspase 3, and β-actin (Calbiochem, San Diego, Calif., USA) for 1 h at room temperature. After being washed with PBS-T, blots were incubated with a 1/5000 dilution of HRP conjugated goat anti-mouse or anti-rabbit IgG for 1 h at room temperature. The protein bands were developed with the ECL Plus Western Blotting Detection System (Pierce).

Total cellular RNA was extracted by an Ultraspec-II RNA isolation system (Biotecx, Houston, Tex., USA) following the manufacturer's instructions. The concentration of RNA was quantified by spectrophotometry at 260 nm. The cDNA in a total volume of 20 μL was prepared after reverse transcription of cellular RNA (5 μg) with Moloney murine leukemia virus reverse transcriptase (Promega, Madison, Wis., USA) using an 20-mer of oligo(dT) as the primer. The cDNA (3 μL) was added to the PCR buffer containing primers at 1.5 μM each, MgCl₂ (1.5 mM), dNTPs (0.2 mM each), and 1 U of Taq DNA polymerase (Promega) in a total reaction volume of 50 μL. The oligonucleotide primers for human Bcl-2, Bax, p53, and β-actin were used according to previously published sequences. Thirty-five cycles were used for Bcl-2, Bax, and p53, and 30 cycles for β-actin (95° C. for 1 min, 55° C. for 2 min, and 72° C. for 3 min) using a PCR controller (GeneAmp PCR System 2400; PerkinElmer, Wellesley, Mass., USA). The PCR products were separated by 1% agarose gel electrophoresis, stained with 0.5 μg mL⁻¹ ethidium bromide, and viewed with UV light.

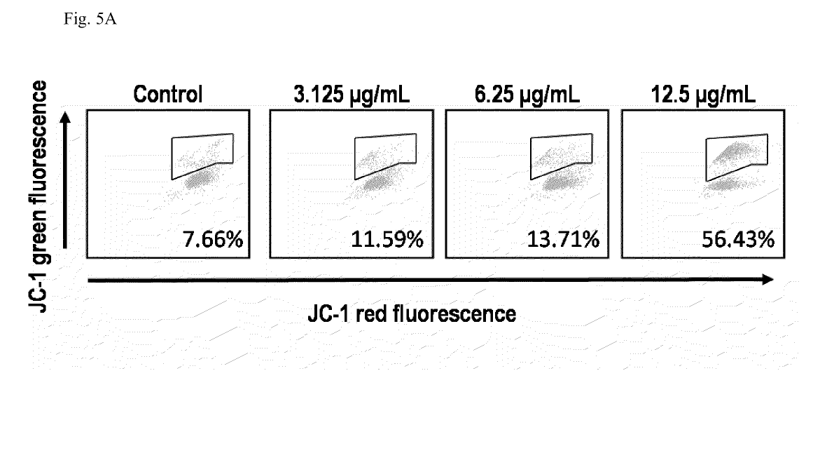

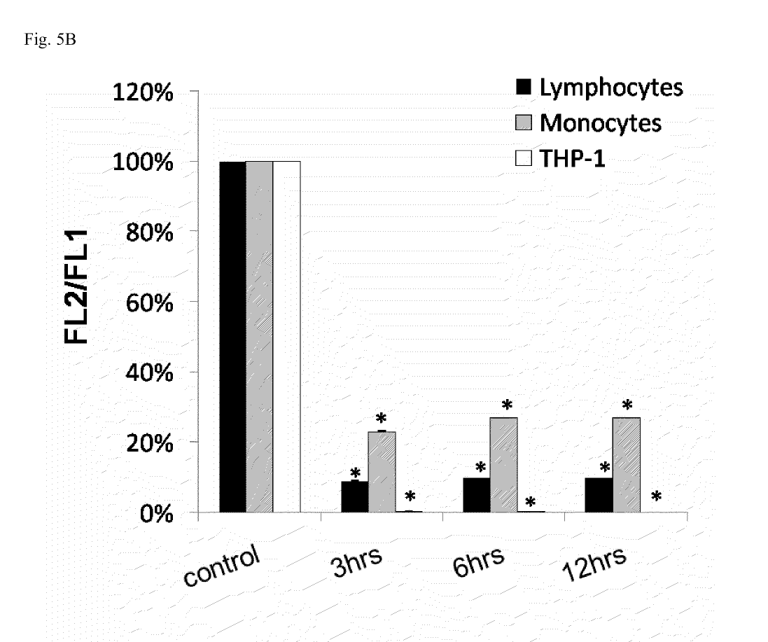

The loss of mitochondrial membrane potential (ΔΨm) is an important event in apoptosis. The carbocyanine dye JC-1 detected changes in ΔΨm due to its dual emission characteristics. A two-parameter fluorescence display of JC-1-stained monocytes revealed that most of the cells emitted relatively lower levels of green fluorescence, whereas a subpopulation exhibited a reduced JC-1 aggregation and a increase in the green fluorescence emission indicating drop of ΔΨm. Exposure of purified monocytes to 3.125 μg mL⁻¹ and 6.25 μg mL⁻¹ of the LcrS₅₋₃₀ for 24 h did not induce a significant change in emission of JC-1 (11.59% and 13.71%, respectively; % of depolarized ΔΨm cells), whereas the 12.5 μg mL⁻¹ LcrS₅₋₃₀ strongly induced depolarized ΔΨm cells (56.43%, P<0.001, FIG. 5A). In addition, a high correlation has been found previously between ΔΨm in isolated mitochondria and fluorescence ratio (mean red fluorescence intensity/mean green fluorescence intensity corresponding to the FL2/FL1 ratio). Therefore, in order to determine the ΔΨm of lymphocytes/monocytes/THP-1 samples after the LcrS₅₋₃₀ treatment; the values of the fluorescence ratio was evaluated (FIG. 5B). The FL2/FL1 ratio was decreased as early as 3 hours after the LcrS₅₋₃₀ treatment. For the tested cells, the maximum loss of ΔΨm was reached at approximately 3 h and remained at this level till 12 h after treatment.

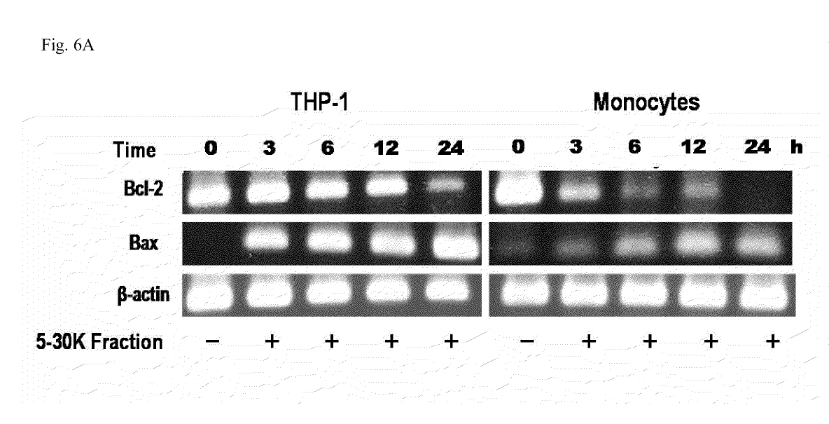

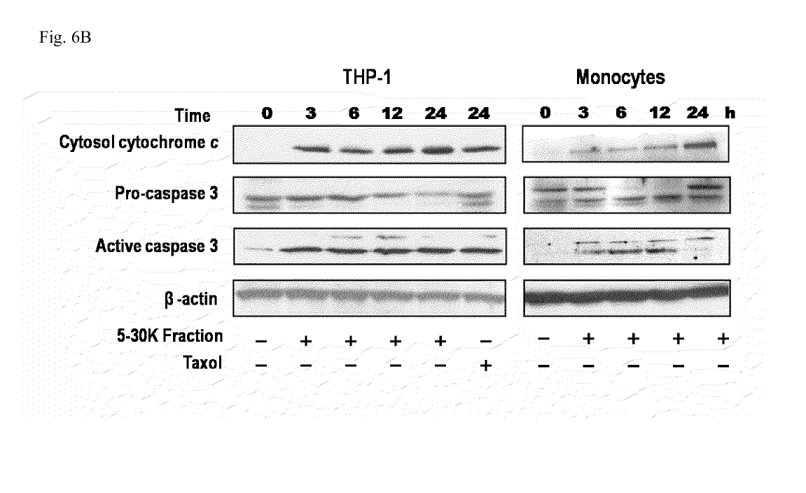

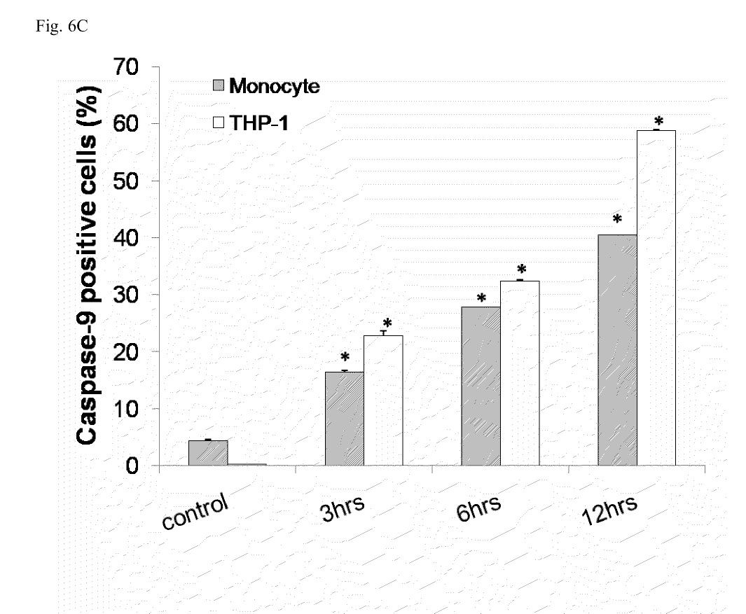

Meanwhile, to understand the modulation in the apoptotic signaling pathway of the LcrS₅₋₃₀, expression of the Bcl-2 family proteins Bcl-2, and Bax were analyzed by the RT-PCR (FIG. 6A). After treatment with the LcrS₅₋₃₀ in the monocytes and in the THP-1 cultures, the mRNA levels of Bcl-2 decreased, whereas that of Bax mRNA expression increased in time dependency (FIG. 6A). The release of the cytochrome c from mitochondrial to cytoplasm was determined by the western blot (FIG. 6B). And, the cleavage of the pro-caspase 9 and pro-caspase 3 into their active form were detected 3 to 24 h after the LcrS₅₋₃₀ treatment (FIGS. 6B & 6C). These results revealed that the role of the LcrS₅₋₃₀ regulated apoptosis of monocytes and the THP-1 cells via expression of both mRNA, such as Bcl-2 and Bax, and proteins, including cytochrome c, caspase 9 and caspase 3, by mitochondria pathway.

### Exp. 7

### Inhibition of LPS-Induced Inflammatory Cytokines Via Treatment of the LcrS₅₋₃₀

Lymphocytes, monocytes and THP-1 cells, 5×10⁵ cells mL⁻¹ were stimulated with 1 μM LPS and 25 μg mL⁻¹ LcrS₅₋₃₀. After 24 h, culture supernatants were collected, centrifuged and stored at −20° C. for cytokines and chemokine analysis. Quantitative human IL-1β, IL-6, IL-8, TNF-α and TGF-01-specific ELISAs (DuoSet, R & D systems, Minneapolis, Minn., USA) were performed in a 96-well plate and detected at 450 nm in the microplate reader (Bio-Rad, Hercules, Calif., USA).

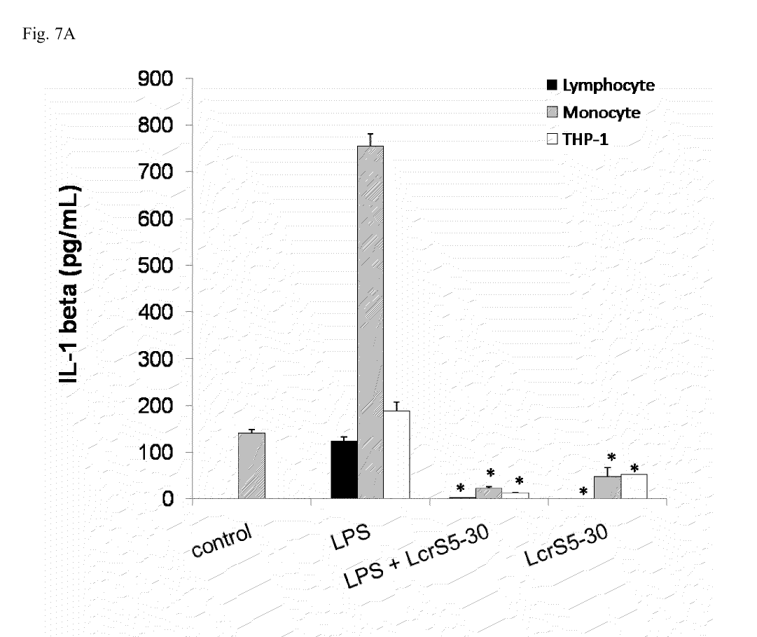

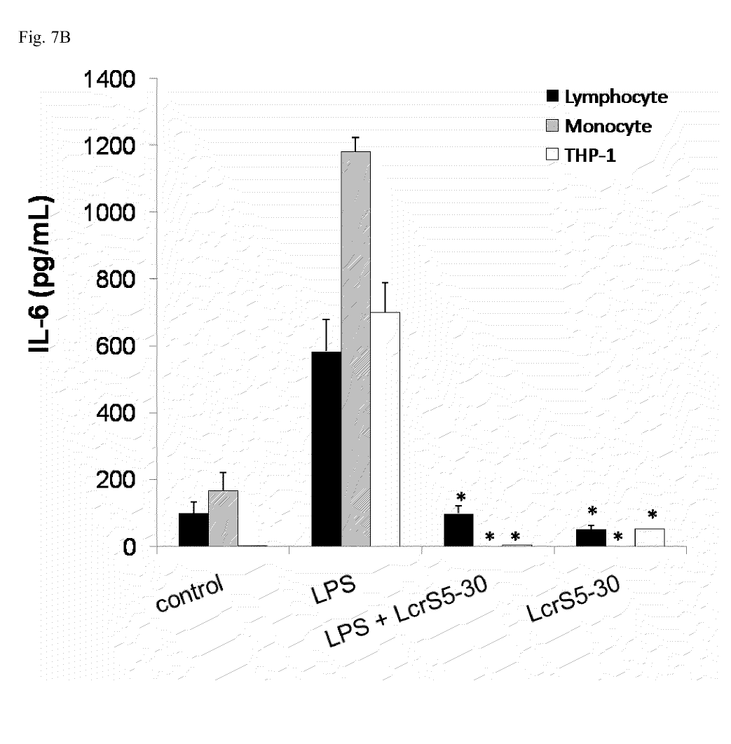

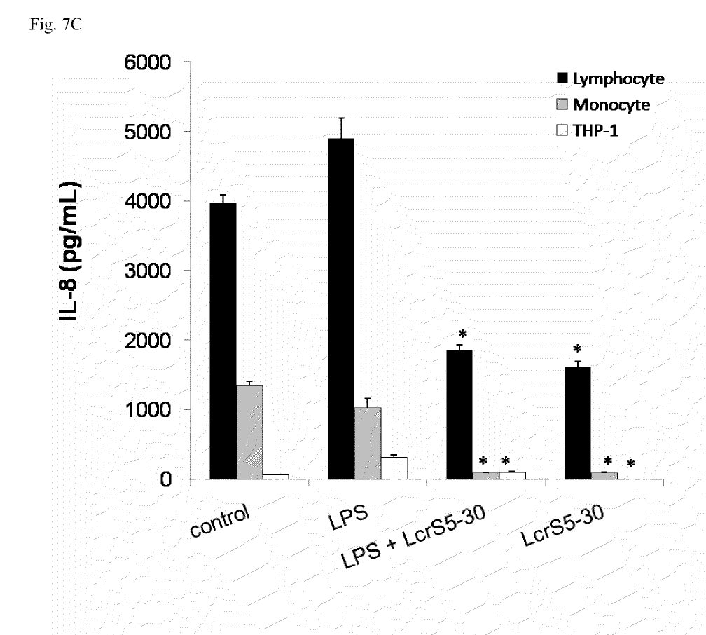

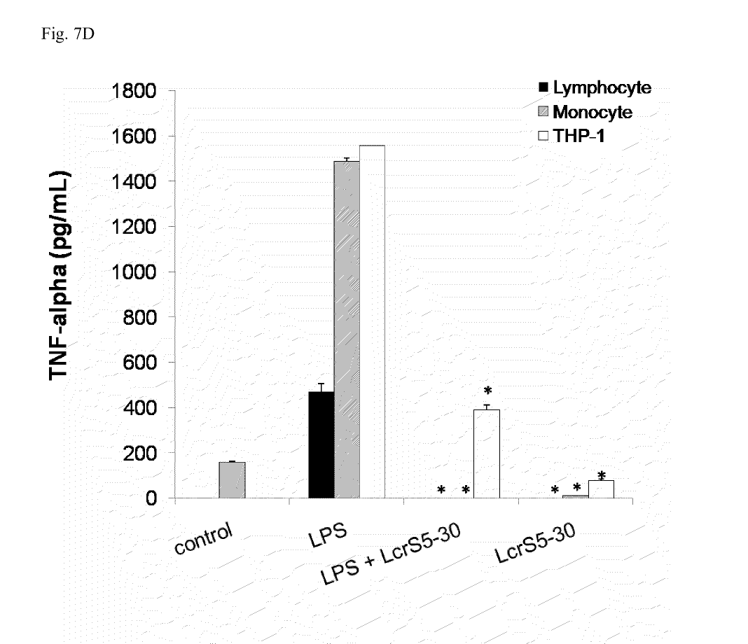

The apoptotic inducing effect on pro-inflammnatory cells by the LcrS₅₋₃₀ implied its potential in regulating the secretion of pro-inflammatory cytokines (IL-1β, IL-6, IL-8 and TNF-α). In order to measure the anti-inflammatory effect, lymphocytes, monocytes and THP-1 cells were treated with E. coli-derived LPS and the LcrS₅₋₃₀, and the changes of inflammatory cytokines secretion were evaluated by ELISA (FIG. 7). As expected, LPS treated cells showed an elevated level of inflammatory cytokines. However, cytokines expression reduced remarkably after coincubation of the LcrS₅₋₃₀ simultaneously (P<0.01, FIG. 7). These experiments suggested that the LcrS₅₋₃₀ was capable of downregulating the secretion of pro-inflammatory cytokines.

### Exp. 8

### The LcrS₅₋₃₀ Induced TGF-β Production and Promoted TGF-β Independent Apoptosis

Lymphocytes, monocytes and THP-1 cells, 5×10⁵ cells mL⁻¹ were stimulated with 1 μM LPS and 25 kg mL⁻¹ LcrS₅₋₃₀. After 24 h, culture supernatants were collected, centrifuged and stored at −20° C. for cytokines and chemokine analysis. Quantitative human IL-1β, IL-6, IL-8, TNF-α and TGF-β1-specific ELISAs (DuoSet, R & D systems, Minneapolis, Minn., USA) were performed in a 96-well plate and detected at 450 nm in the microplate reader (Bio-Rad, Hercules, Calif., USA).

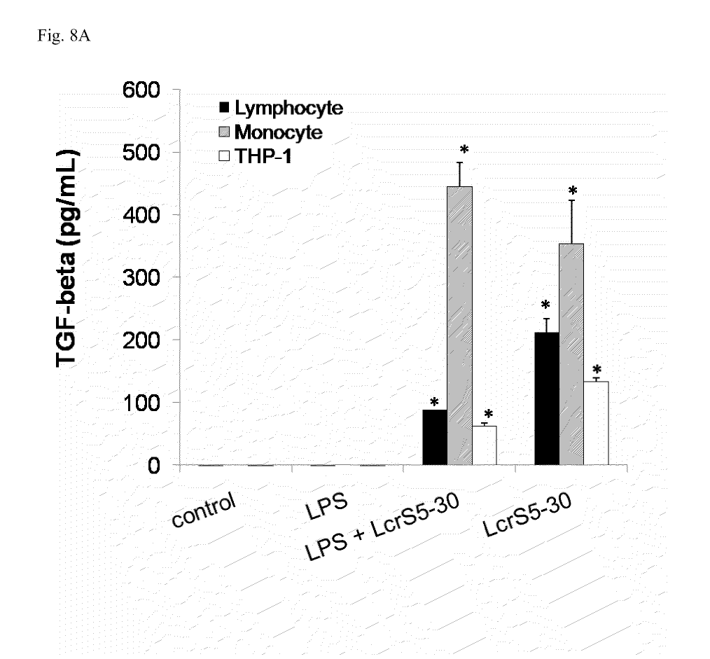

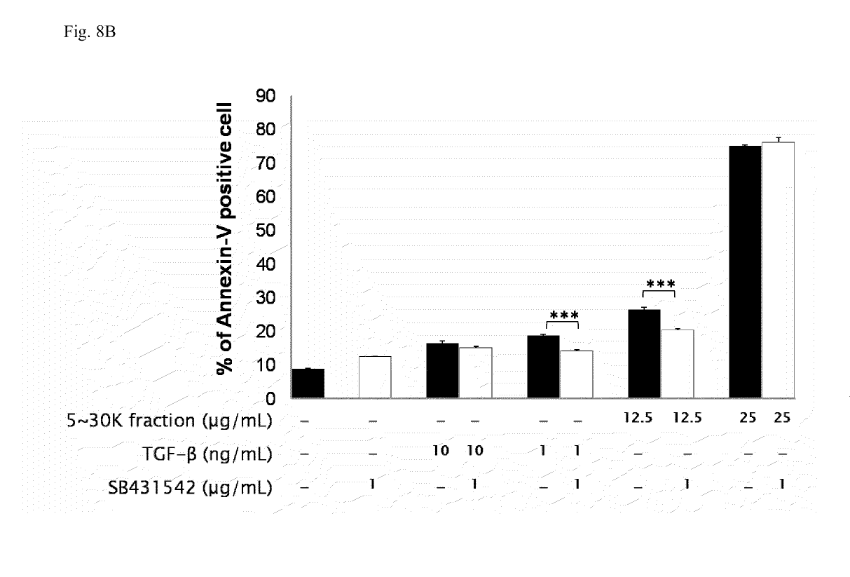

TGF-β1 is a key regulatory cytokine involved in anti-inflammation that counteracts IL-1 and TNF-α modulates processes such as homing, cellular adhesion, chemotaxis, and T-cell homeostatic regulation. Different from pro-inflammatory cytokines as mentioned above, drastic increase of TGF-β1 level was observed following treatment of the LcrS₅₋₃₀ (FIG. 8A).

The TGF-β regulates a wide variety of biological functions including apoptosis. The TGF-β production was increasing in the LcrS₅₋₃₀ treated THP-1 cells. It was reasonable to postulate that apoptosis might be triggered by TGF-β rather than the LcrS₅₋₃₀. To confirm this assumption, a pharmacological inhibitor, SB431542, would be applied. The percentages of apoptotic cells in 10 ng mL⁻¹ and 1 ng mL⁻¹ TGF-β exposed preparations were 18.9±0.44% and 16.57±0.67%, respectively (FIG. 8B). Treatment with 12.5 μg mL⁻¹ or 25 μg mL⁻¹ of the LcrS₅₋₃₀ induced dissimilar level of apoptosis of THP-1 cells that were 26.35±0.77% and 74.96±0.38%, respectively. One ng mL⁻¹ TGF-β and 12.5 μg mL⁻¹ the LcrS₅₋₃₀ induced apoptosis were SB431542 sensitive. Nevertheless, 25 μg mL⁻¹ of LcrS₅₋₃₀ induced drastic change on the level of apoptosis on THP-1 cells and SB431542 could not reverse this phenomenon (FIG. 8B). Collectively these data indicated that the LcrS₅₋₃₀ was capable of inducing TGF-β independent cell death.

What is claimed is:

1. A composition for inhibiting gastrointestinal inflammation comprising an effective amount of Lactoacillus casei rhamnosus secreted factors 5 to 30K fraction.

2. The composition of claim 1, wherein the secreted factors of L. casei rhamnosus alters pro-inflammatory or anti-inflammatory cytokines balance to treat gastrointestinal inflammation.

3. A method of preventing gastrointestinal inflammation comprising administrating a subject in effectively amount of the extract of L. casei rhamnosus secreted factors 5 to 30K fraction.

4. The method of claim 3, wherein the subject is human.

5. The method of claim 3, wherein said preventing inflammatory is by promoting inflammatory cells apoptosis

6. The method of claim 5, wherein the inflammatory cells are monocytes, lymphocytes and human monocytic leukemia-derived cells (THP-1).

7. The method of claim 5, wherein the apoptosis is induced by mitochondrial pathway.

8. A method of suppressing pro-inflammatory cytokine production comprising administrating a subject an effectively amount of the extract of claim 3.

9. The method of claim 8, wherein the subject is human.

10. The method of claim 8, wherein the pro-inflammatory cytokine is induced by lipopolysaccharide (LPS) in activated cells of lymphocytes, primary monocyte or human monocytoid cell lines (THP-1).

11. The method of claim 8, wherein the pro-inflammatory cytokine is (a) IL-1β, (b) IL-6, (c) TNF-α, (d) chemokine, or (e) IL-8 at least.

12. The method of claim 8, wherein suppressing pro-inflammatory cytokine concomitantly suppresses lymphocyte, monocyte, and THP-1 chemotaxis and cellular activation.

13. A method of suppressing activation and proliferation of inflammatory cells comprising administrating a subject an effectively amount of the extract of claim 1.

14. The method of claim 13, wherein the subject is human.

15. The method of claim 13, wherein the suppressing activation or proliferation of inflammatory cells is by enhancing TGF-β1 level.

16. The method of claim 15, wherein the TGF-β1 is an anti-inflammatory cytokine with a vital role in suppressing the activation and proliferation of inflammatory cells.

17. The method of claim 15, wherein the inflammatory cells is lymphocytes, monocytes, or THP-1.
